(19) United States
(12) Patent Application Publication
(10) Pub. No.: US 2025/0099013 A1
(43) Pub. Date: Mar. 27, 2025

(54) BIO NEUROFEEDBACK MANAGEMENT SYSTEM FOR EARLY, REAL-TIME DETECTION OF STRESS-INDUCED BIOMARKERS RELATED TO NEUROLOGICAL AND PSYCHOLOGICAL DISORDERS

(71) Applicant: Cogito LLC, Greenville, SC (US)

(72) Inventors: Brandon Williams, Greenville, SC (US); Nicholas Boltin, Columbia, SC (US); Yogesh Rana, Greenville, SC (US); Samuel Yeboah, Greenville, SC (US)

(73) Assignee: Cogito LLC, Greenville, SC (US)

(21) Appl. No.: 18/373,429

(22) Filed: Sep. 27, 2023

(51) Int. Cl. A61B 5/369; A61B 5/00; A61B 5/0205; A61B 5/024; A61B 5/08

(52) U.S. Cl. CPC A61B 5/369; A61B 5/0205; A61B 5/6803; A61B 5/6805; A61B 5/02438; A61B 5/0816

(57) ABSTRACT

Methods and systems for collecting, cataloging, and analyzing brainwaves and psychological activity utilize mobile biomonitoring devices and clinical decision support tools to monitor neurological responses for early detection of trauma-related neurological disorders.

### BACKGROUND OF THE DISCLOSURE

Electroencephalography (EEG), functional magnetic resonance imaging (fMRI), and other neural-interface devices are used by physicians and neuroscientists to monitor biopotential brainwaves and psychological activities. These devices help diagnose neurological conditions such as sleep disorders, epilepsy, and Alzheimer's, as well as psychological disorders related to traumatic events such as Acute Stress Disorder (ASD) and Posttraumatic Stress Disorder (PTSD), without invasive surgical procedures. Unfortunately, known diagnostic equipment is expensive, cumbersome, and complex. Hospitals can spend several million dollars on a single fMRI and hundreds of thousands more to operate annually; thus, clinical EEG and fMRI devices tend to be in limited supply due to their costs. In addition, the clinical evaluation process for diagnosing mental health disorders like ASD is based on history and physical examination within days following a traumatic incident. Screenings and questionaries are the most common diagnostic tools that require significant training and often miss initial psychological symptoms. Misdiagnosis and/or improper treatment may lead to a person feeling detachment, reduced awareness, derealization, depersonalization, and/or dissociative amnesia. Still further, known diagnostic procedures lack Artificial Intelligence (AI) and data-driven decision support.

What is needed in the industry is a non-invasive, portable system that can be worn or carried by a person for monitoring brain activity in real-time during normal daily activities to detect early signs of trauma-related neurological disorders.

### SUMMARY OF THE DISCLOSURE

The present disclosure provides systems and methods for collecting, cataloging, and analyzing brainwaves and neurological activity using, for instance, a non-invasive EEG cap and/or other wearable gear. Related hardware can be superimposed or paired with photo-emitting devices to permit alteration in photo-electric light or activity based on the monitored brain waves, more particularly, for the purpose of establishing data pipelines for superimposed AI-driven decision support systems. More particularly, a comprehensive neurofeedback management system that includes a mobile EEG biomonitoring device and a clinical decision support tool designed to monitor neurological responses continuously powered by AI detect early signs of trauma-related neurological disorders. By identifying EEG-associated neurological biomarkers in the early stages, effective mitigation strategies can be used to reduce the risk of neurological and psychological disorders.

In one embodiment according to the disclosure, an exemplary method of monitoring biomarkers in an individual may include monitoring a monitored individual with at least one sensing device disposed proximate to the monitored individual, wherein the sensing device is operable to sense electroencephalographic signals of the monitored individual; sending the electroencephalographic signals to a monitoring system; comparing the electroencephalographic signals against a normalized profile for the monitored individual to assess in real-time whether the monitored individual is in neuropsychological distress and, sending an alert from the monitoring system regarding the monitored individual. Glasses, a helmet, headgear, and/or a vest may be provided to carry the sensing device or in which the sensing device is installed.

The vest, for instance, in this embodiment may include a control unit in electrical communication with the sensing device. Further, the vest may be equipped with a heart rate monitor and/or a respiration monitor in electrical communication with the monitoring system.

This embodiment may also include a module operable to sense a heart rate and/or to sense respiration and may further include displaying the electroencephalographic signals in a graphic display.

In another exemplary embodiment, a system for monitoring biomarkers of an individual may include a sensing device operable to sense an electroencephalographic signal of a monitored individual; a receiver configured to receive the electroencephalographic signal of the monitored individual; a database for interpreting the electroencephalographic signal received from the receiver; at least one computing device to interpolate the electroencephalographic signal against a normalized profile to determine whether the monitored individual is in biometric danger; and a transmitter configured for sending an alert the monitored individual if the individual is in biometric danger.

In yet another embodiment, a method for post-processing biomarker signals collected from a biomonitoring device may include attaching a biomonitoring device to a subject, the biomonitoring device in communication with biomarkers produced by the subject; obtaining biomarker data from the subject; formatting the biomarker data into storable files; classifying the biomarker data into biopotential waveforms; normalizing the biopotential waveforms; and determining trends from the biopotential waveforms as predictors of stress. The biomonitoring device may be wearable by the subject, and the resulting biomarker data may include EEG waveforms.

Additional objects and advantages of the present subject matter are set forth in, or will be apparent to, those of ordinary skill in the art from the description herein. Also, it should be further appreciated that modifications and variations to the specifically illustrated, referenced, and discussed features, processes, and elements hereof may be practiced in various embodiments and uses of the disclosure without departing from the spirit and scope of the subject matter. Variations may include, but are not limited to, substitution of equivalent means, features, or steps for those illustrated, referenced, or discussed, and the functional, operational, or positional reversal of various parts, features, steps, or the like. Those of ordinary skill in the art will better appreciate the features and aspects of the various embodiments, and others, upon review of the remainder of the specification.

### BRIEF DESCRIPTION OF THE DRAWINGS

A full and enabling disclosure of the present subject matter, including the best mode thereof directed to one of ordinary skill in the art, is set forth in the specification, which refers to the appended figures, wherein:

FIG. 1 is a front elevational view of a Bio-Neurofeedback Management System (“BNMS”) being worn in an intended use application according to an embodiment of the disclosure;

FIG. 2 is a rear elevational view of the BNMS as in FIG. 1;

FIG. 3 is a side elevational view of the BNMS as in FIG. 1;

FIG. 4 is a top perspective view of a BNMS according to another embodiment of the disclosure;

FIG. 5 is a diagrammatic representation of an operation of the BNMS as in FIG. 4;

FIG. 6A is a front elevational view of a BNMS according to another embodiment of the disclosure, particularly showing heart rate and respiratory modules in an enlarged inset;

FIG. 6B is a side elevational view of the BNMS as in FIG. 6A, particularly showing a power supply and a control unit in an enlarged inset;

FIG. 6C is a rear elevational view of the BNMS as in FIG. 6A, particularly showing power and control wiring in a partial, sectional, enlarged inset;

FIG. 7A is a front elevational view of a BNMS according to another embodiment of the disclosure;

FIG. 7B is a side elevational view of the BNMS as in FIG. 7A;

FIG. 7C is a rear elevational view of the BNMS as in FIG. 7A;

FIG. 8 is a rear elevational view of the BNMS in connection with FIG. 7C;

FIG. 9 is a front elevational view of the BNMS as in FIG. 8, particularly showing heart rate and respiratory modules in an intended use;

FIG. 10 is a side elevational view of the BNMS as in FIG. 9;

FIG. 11 is a circuit diagram showing an electrical interoperation of a BNMS according to another aspect of the disclosure;

FIG. 12 is a flowchart showing an exemplary process for normalizing, determining trends, and visualizing values based on the BNMS embodiments according to another aspect of the disclosure;

FIGS. 13A and 13B shows EEG test results utilizing BNMS embodiments according to aspects of the disclosure;

FIG. 14 shows biopotential waveforms for predicting stressful events utilizing BNMS embodiments according to aspects of the disclosure; and

FIG. 15 is a graph of Artificial Neural Network (ANN) data utilizing BNMS embodiments according to aspects of the disclosure.

### DETAILED DESCRIPTION OF THE DISCLOSURE

As required, detailed embodiments are disclosed herein; however, the disclosed embodiments are merely exemplary and may be embodied in various forms. Therefore, specific structural and functional details disclosed herein are not to be interpreted as limiting, but merely as a basis for the claims and as a representative basis for teaching one skilled in the art to variously employ the exemplary embodiments of the present disclosure, as well as their equivalents.

Unless defined otherwise, all technical and scientific terms used herein have the same meaning as is commonly understood by one of ordinary skill in the art to which this disclosure belongs. In the event that there is a plurality of definitions for a term or acronym herein, those in this section prevail unless stated otherwise.

“Artificial Intelligence” (AI) means a synthetic entity that can make decisions, solve problems, and function like a human being by learning from examples and experience, understanding human language, and/or interactions with a human user, i.e., via a chat system. The AI synthetic entity may be equipped with memory and a processor having a neural network, as well as other components, which can iteratively learn via supervised machine learning (ML) (for example, through inputted data) or capable of autonomous, unsupervised deep learning (DL) (for example, based on inputted data or perceived data and trial and error). AI, ML, and DL may be used interchangeably herein.

“BNMS” means a Bio-Neurofeedback Management System for early, real-time detection of stress-induced biomarkers, including but not limited to neurological and psychological disorders.

“Computing Device,” “User,” or “User Device” means any portable, non-portable, wearable, non-wearable, embedded, non-embedded, automated, human controlled device, or software that can access the World Wide Web (the “Internet”).

“DC” means a monitoring or data center.

“Frame” is a container for a single Packet pursuant to an OSI (Open Systems Interconnection) model.

“Internet Capable Device” means including but not limited to portable, non-portable, wearable, non-wearable, embedded, non-embedded, automated, human controlled devices, or software and combinations thereof, capable of using a proxy.

“Latency” (or Lag) is a time delay between a cause and an effect of some physical change in the system being observed, but as used herein “latency” is a time interval between the input to a stimulation and the visual or auditory response, often occurring because of network delay.

“Monitored person,” “monitored individual,” “end user,” “patient,” “subject,” or the like means someone who is utilizing a BNMS.

“Multihoming” means the practice of connecting a host or a computer network to more than one network to increase reliability or performance.

“Neural network” means AI having an input level or data entry layer, a processing level (which includes at least one algorithm to receive and interpret data but generally at least two algorithms that process data by assigning significances, biases, et cetera to the data and interact with each other to refine conclusion or results), and an output layer or results level that produces conclusions or results.

“Packet” is a Formatted Unit of Data.

Wherever the phrases “for example,” “such as,” “including,” and the like are used herein, the phrase “and without limitation” is understood to follow unless explicitly stated otherwise. Similarly, “an example,” “exemplary,” and the like are understood to be non-limiting.

The term “substantially” allows for deviations from the descriptor that do not negatively impact the intended purpose. Descriptive terms are understood to be modified by the term “substantially” even if the word “substantially” is not explicitly recited.

The term “about” when used in connection with a numerical value refers to the actual given value, and to the approximation to such given value that would reasonably be inferred by one of ordinary skill in the art, including approximations due to the experimental and or measurement conditions for such given value.

Unless the context clearly requires otherwise, throughout the description and the claims, the words “comprise”, “comprising”, and the like are to be construed in an inclusive sense as opposed to an exclusive or exhaustive sense; in the sense of “including, but not limited to.”

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, et cetera. Thus, for example, “a device having components a, b, and c” means that the device includes at least components a, b, and c. Similarly, the phrase: “a method involving steps a, b, and c” means that the method includes at least steps a, b, and c.

Where a list of alternative component terms is used, e.g., “a structure such as ‘a’, ‘b’, ‘c’, ‘d’ or the like”, or “a” or b”, such lists and alternative terms provide meaning and context for the sake of illustration, unless indicated otherwise. Also, relative terms such as “first,” “second,” “third,” “front,” and “rear” are intended to identify or distinguish one component or feature from another similar component or feature, unless indicated otherwise herein.

The various embodiments of the disclosure and/or equivalents falling within the scope of the present disclosure overcome or ameliorate at least one of the disadvantages of the prior art.

Detailed reference will now be made to the drawings in which examples embodying the present subject matter are shown. The detailed description uses numerical and letter designations to refer to features of the drawings. The drawings and detailed description provide a full and written description of the present subject matter, and of the manner and process of making and using various exemplary embodiments, so as to enable one skilled in the pertinent art to make and use them, as well as the best mode of carrying out the exemplary embodiments. The drawings are not necessarily to scale, and some features may be exaggerated to show details of particular components. Thus, the examples set forth in the drawings and detailed descriptions are provided by way of explanation only and are not meant as limitations of the disclosure. The present subject matter thus includes any modifications and variations of the following examples as come within the scope of the appended claims and their equivalents.

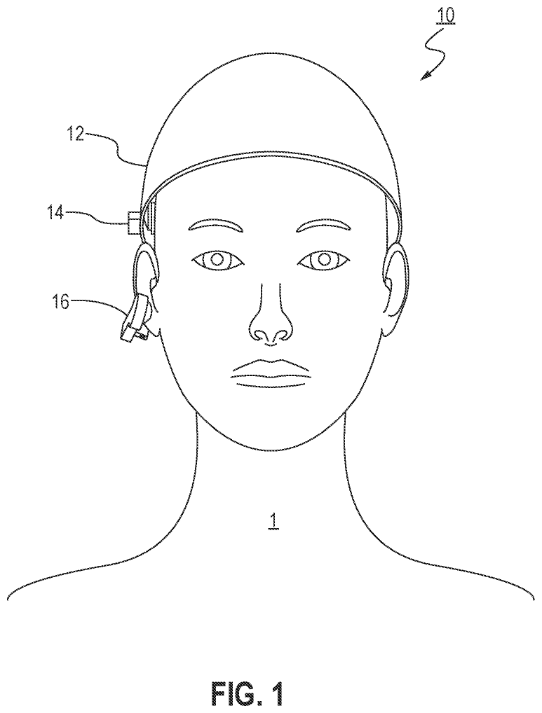

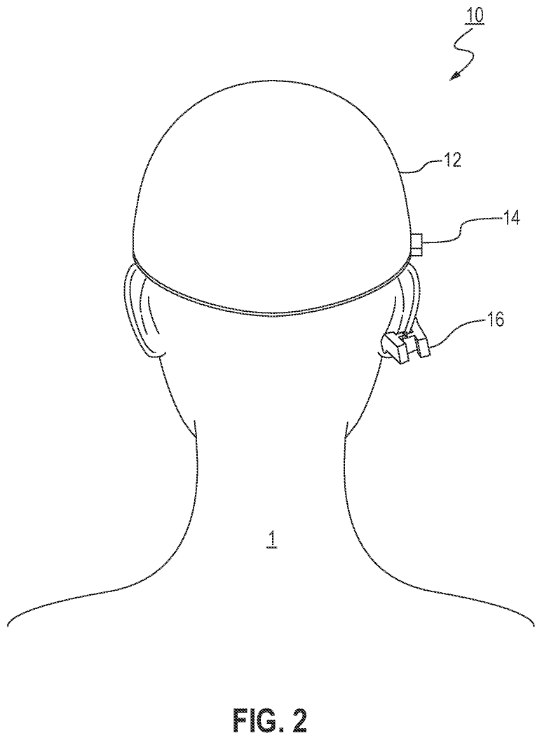

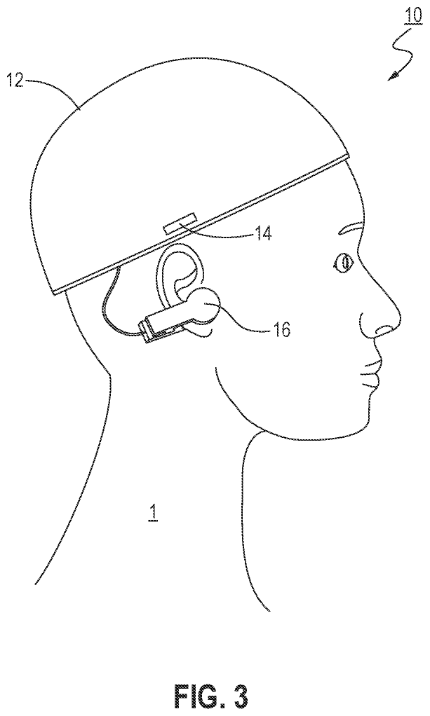

Turning now to FIGS. 1 through 3, a Bio-Neurofeedback Management System (“BNMS”) according to one embodiment of the disclosure is broadly designated by the element number 10. In this example, the BNMS 10 is worn as a cap or headgear 12 by a user or patient 1 and includes an EEG sensor 14 that further includes a blood flow device 16 to complete the brain activity waveform circuit as explained in greater detail below.

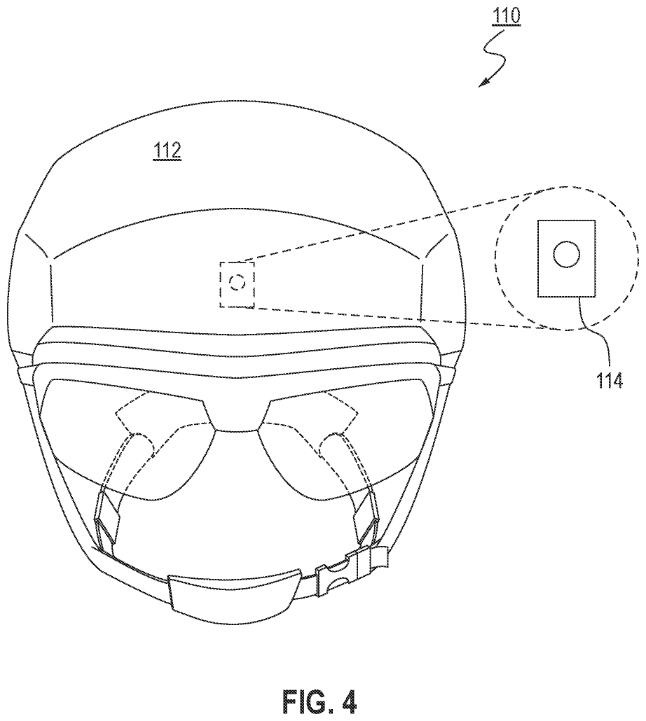

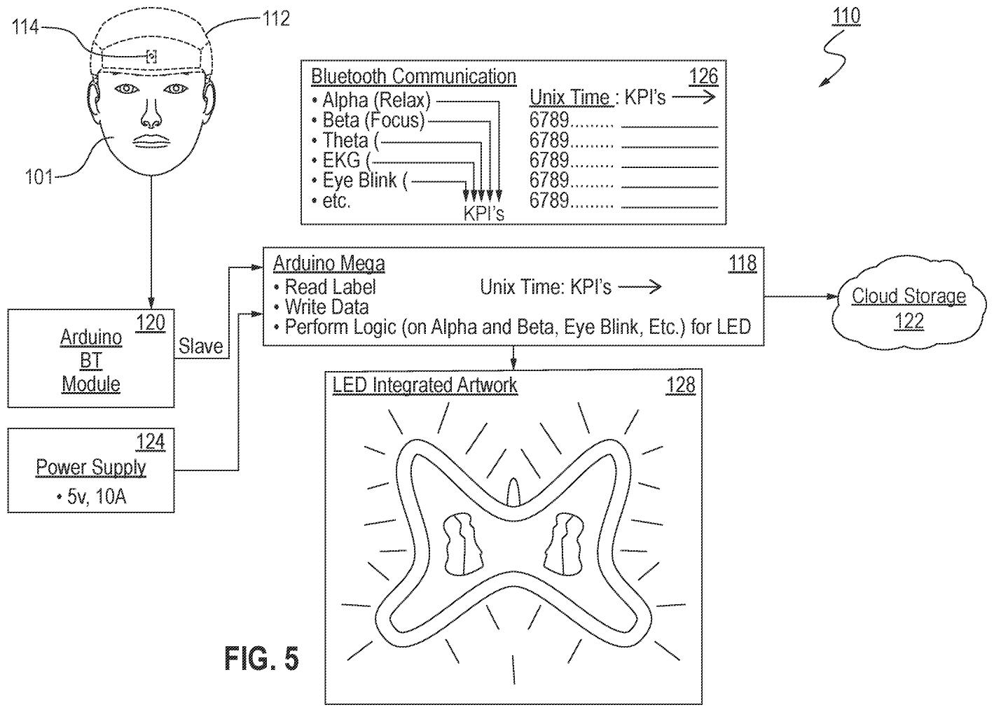

FIG. 4 shows another embodiment of a BNMS 110 which employs a helmet 112 equipped with an EEG sensor 114. The helmet 112 may be worn, for instance, by a soldier 101 (see FIG. 5) in combat or during a training exercise to monitor brain activity and send an alert to a remote site or monitoring station (schematically depicted as element number 122 in FIG. 5) regarding possible life-threatening overexertion or combat fatigue.

FIG. 5 shows that the EEG sensor 114 may be wirelessly connected to a remote computer database 118 using, for instance, an interface module 120 installed in the helmet 112. The interface module 120 may utilize a Bluetooth® wireless system or other wireless capability. Further, the EEG sensor 114 and the interface module 120 may be powered by a power supply 124, such as a rechargeable battery.

By way of example, FIG. 5 further shows that the EEG sensor 114 transmits brainwave information 126 through the wireless interface 120 to the database 118. More particularly, the information 126 may include alpha, beta, and theta waveforms as well as EKG information. The database 118 reads the received data, performs logic functions, and may produce an integrated map or presentation 128 of the brainwave and other psychological conditions of the soldier 101. For instance, one set of test data showed the strongest positive correlation between GammaMid with GammaLow, BetaHigh with BetaLow, and AlphaHigh with AlphaLow with 0.77, 0.64, and 0.43, respectively. Alpha waveforms are typically in the range of 8-12 Hz, with AlphaLow and AlphaHigh being 8-10 Hz and 11-12 Hz, respectively. The Delta and Theta waveforms showed the lowest correlation with any other biopotential ranging from −0.13 to 0.11 and 0.09 to 0.18, respectively. Delta and Theta waveforms are associated with sleep. While awake, therefore, inordinate activity revealed by the integrated map 128 may suggest a departure from a normalized profile for a monitored individual and may further suggest an abnormal condition such as a neurological disorder, e.g., Acute Stress Disorder (ASD) that could be a precursor to Post Traumatic Stress Disorder (PTSD).

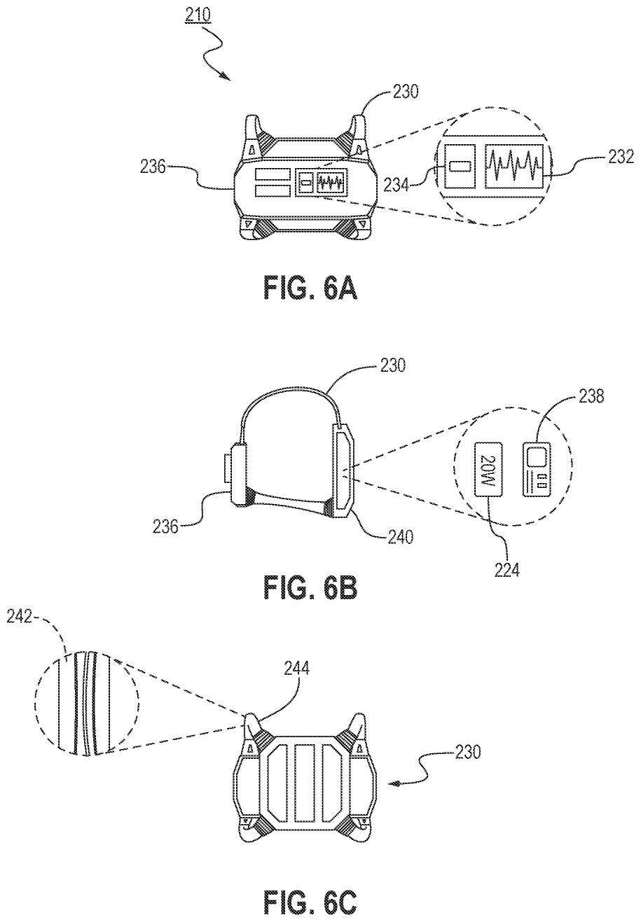

With reference now to FIGS. 6A, 6B, and 6C, another aspect of a BNMS 210 is shown integrated in combat body armor that can be worn as vest or backpack 230. In this example, a heart rate monitor, sensor, or module 232 and a respiratory monitor, sensor, or module 234 may be carried by a front portion or strap 236 of the vest 230. Also shown, a power supply 224, such as a lithium ion battery, and a control unit 238 may be installed in a rear portion 240 of the vest 230. Here, power and control wiring 242 may be located within straps 244 of the vest 230 thereby connecting the heart rate and respiratory modules 232, 234 to the power supply 224 and the control unit 238.

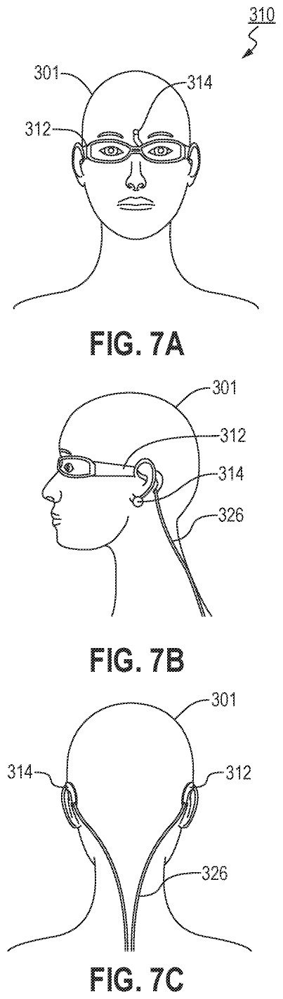

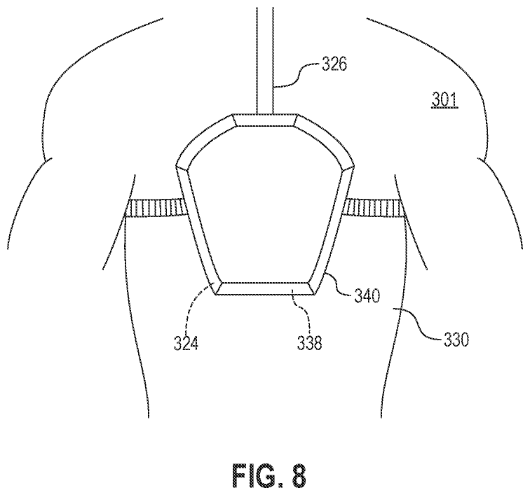

FIGS. 7A through 10 show another exemplary embodiment of a BNMS 310. As shown in FIGS. 7A through 7C, a monitored individual 301 wears EEG smart glasses 312 that are equipped with an EEG sensor 314. The sensor 314 is in electrical communication with a vest 330 worn by the individual 301 as shown in FIG. 8, which also indicates a power supply 324 and a control unit 338 embedded in a rear portion 340 of the vest 330.

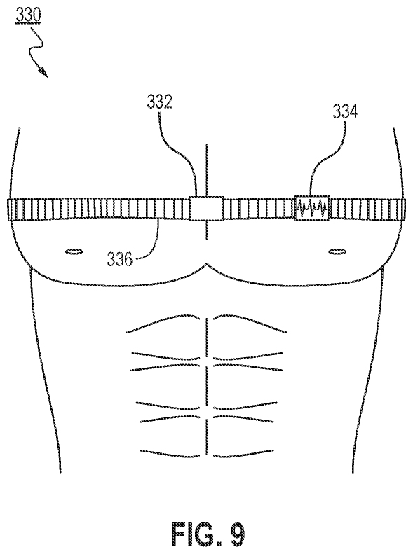

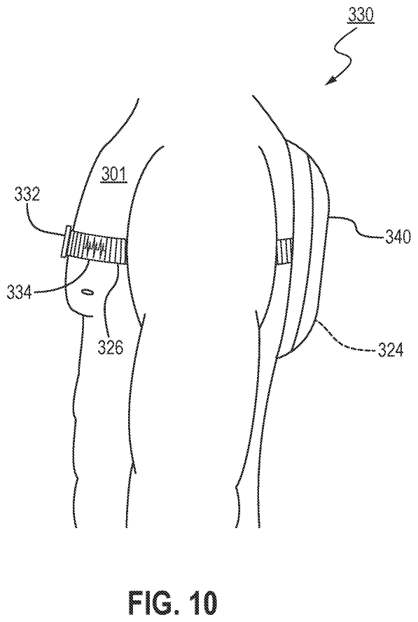

FIGS. 9 and 10 most clearly show a heart rate monitor, sensor, or module 332 and a respiratory monitor, sensor, or module 334 carried by a front portion or strap 336 of the vest 330. As shown, the modules 332, 334 may be in direct contact with the skin of the monitored individual 301 to provide most efficient data readings.

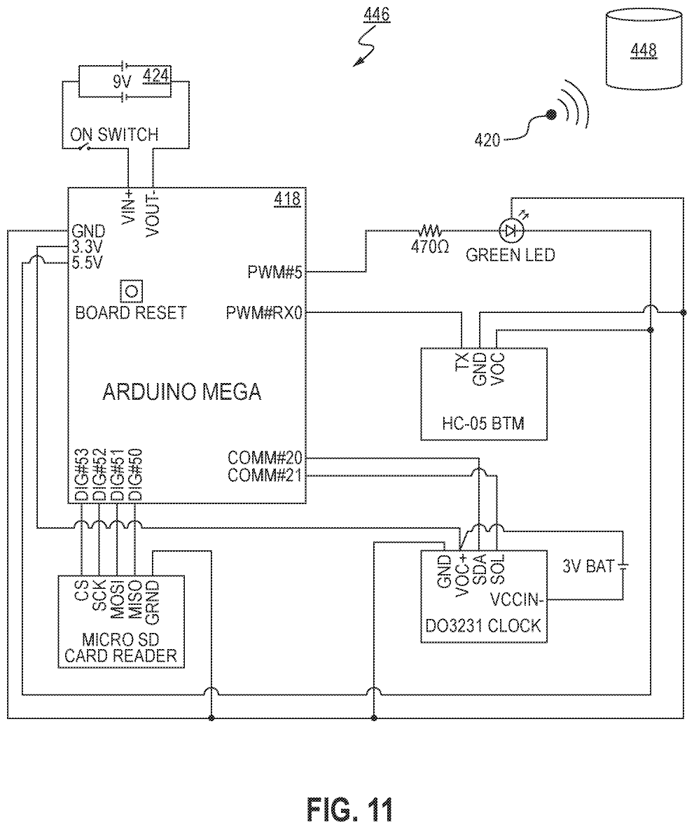

FIG. 11 is a circuit diagram or schematic of a control board, which is broadly designated by the element number 446, that may be employed by various embodiments disclosed herein. The circuit diagram 446 (also referred to herein as BNMS sensor circuit) depicts a principle of operation of an EEG sensor system, such as those within the BNMS embodiments described above. Here, the BNMS sensor circuit 446 provides an analog voltage input 424 of, e.g., 9V to a microcontroller unit (MCU) 418, such as an Arduino® Mega board. The unit 418 receives biometric data from an EEG sensor installed in a cap, helmet, glasses, and the like in physical contact with a user. The BNMS sensor circuit 446 may be in wireless communication 420 with an interface module carried by the cap, helmet, glasses, and the like for transmitting the received data to a remote computer, monitor, monitoring system, monitoring station 448 for analysis, interpolation, or immediate action, such as removal of an individual from a developing ASD situation.

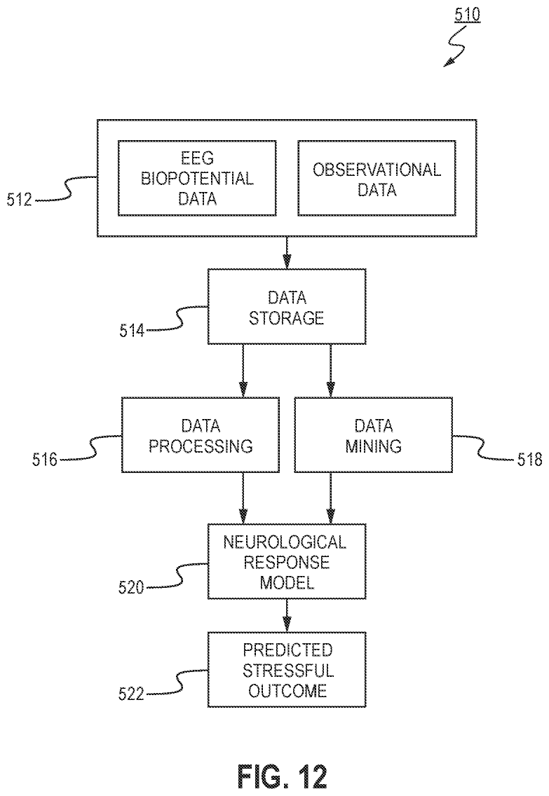

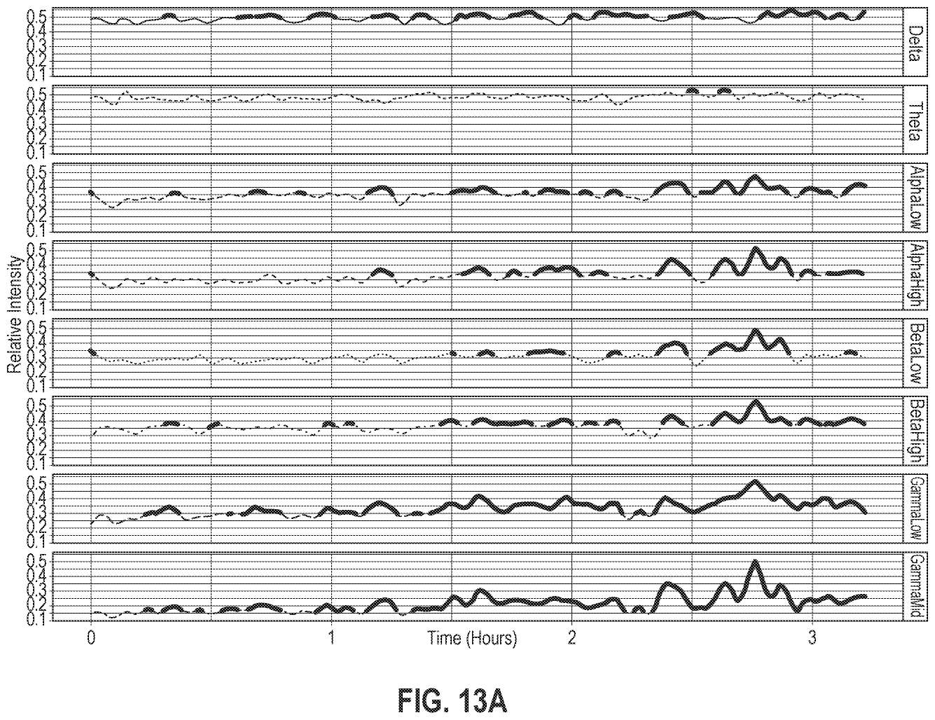

FIG. 12 shows an exemplary method, broadly indicated by element number 510, for post-processing raw EEG signals at step 512 collected from biomonitoring devices such as the BNMS embodiments described herein. The coded methods may include storing at step 514, reading/processing at step 516, and formatting raw data files for mining the data contained therein at step 518. The coded methods may further include classification of biopotential waveforms and modeling those waveforms at step 520, and normalizing, determining trends, and visualizing values indicated at step 522 to predict outcomes.

More particularly, the comprehensive neurofeedback management system utilized in FIG. 12 may include a mobile EEG biomonitoring device and a clinical decision support tool designed to monitor neurological responses continuously powered by AI, which can detect early signs of trauma-related neurological disorders. By identifying EEG-associated neurological biomarkers in early stages, effective mitigation strategies can be used to reduce the risk of neurological and psychological disorders.

By way of example, the following Table 1 shows an Arduino® Bluetooth® Interface with Mindwave® brainwave function that is triggered when brain activity of the user 1 as in FIG. 1 is being monitored by the BNMS 10.

```
TABLE 1
  byte ReadOneByte( ) {
    int ByteRead;
    // Wait until there is data
    while(!Serial.available( ));
    //Get the number of bytes (characters) available for reading from the serial
port.
    //This is data that is already arrived and stored in the serial receive buffer
(which holds 64 bytes)
    ByteRead = Serial.read( );
      return ByteRead; // read incoming serial data
    }
unsigned int delta_wave = 0;
unsigned int theta_wave = 0;
unsigned int low_alpha_wave = 0;
unsigned int high_alpha_wave = 0;
unsigned int low_beta_wave = 0;
unsigned int high_beta_wave = 0;
unsigned int low_gamma_wave = 0;
unsigned int mid_gamma_wave = 0;
void read_waves(int i) {
    delta_wave = read_3byte_int(i);
    i+=3;
    theta_wave = read_3byte_int(i);
    i+=3;
    low_alpha_wave = read_3byte_int(i);
    i+=3;
    high_alpha_wave = read_3byte_int(i);
    i+=3;
    low_beta_wave = read_3byte_int(i);
    i+=3;
    high_beta_wave = read_3byte_int(i);
    i+=3;
    low_gamma_wave = read_3byte_int(i);
    i+=3;
    mid_gamma_wave = read_3byte_int(i);
}
int read_3byte_int(int i) {
return ((payloadData[i] << 16) + (payloadData[i+1] << 8) + payloadData[i+2]);
}
void loop( ) {
    // Look for sync bytes
    // Byte order: 0xAA, 0xAA, payloadLength, payloadData,
    // Checksum (sum all the bytes of payload, take lowest 8 bits, then bit inverse
on lowest
if(ReadOneByte( ) == 0xAA) {
if(ReadOneByte( ) == 0xAA) {
payloadLength = ReadOneByte( );
if(payloadLength > 169) //Payload length cannot be greater than 169
return;
payloadChecksum = 0;
    for(int i = 0; i < payloadLength; i++) { //loop until payload length
is complete
    payloadData[i] = ReadOneByte( ); //Read payload
    payloadChecksum += payloadData[i];
}
checksum = ReadOneByte( ); //Read checksum byte from
stream
    payloadChecksum = 255 − payloadChecksum; //Take one's compliment of
generated checksum
    if(checksum == payloadChecksum) {
      poorQuality = 200;
      attention = 0;
      meditation = 0;
}
    brainwave = false;
    for(int i = 0; i < payloadLength; i++) { // Parse the payload
      switch (payloadData[i]) {
      case 02:
      i++;
      poorQuality = payloadData[i];
      bigPacket = true;
      break;
    case 04:
      i++;
      attention = payloadData[i];
      break;
    case 05:
      i++;
      meditation = payloadData[i];
      break;
    case 0x80:
      i = i + 3;
      break;
```

| | |
|---|---|
| case 0x83: | // ASIC EEG POWER INT |

```
      i++;
      brainwave = true;
      byte vlen = payloadData[i];
      //mySerial.print(vlen, DEC);
      //mySerial.println( );
      read_waves(i+1);
      i += vlen; // i = i + vlen
      break;
```

| | |
|---|---|
| } | // switch |
| } | // for loop |

```
if(bigPacket) {
    if(poorQuality == 0){
    }
```

| | |
|---|---|
| else{ | // do nothing |

```
      }
    }
```

### Test Results

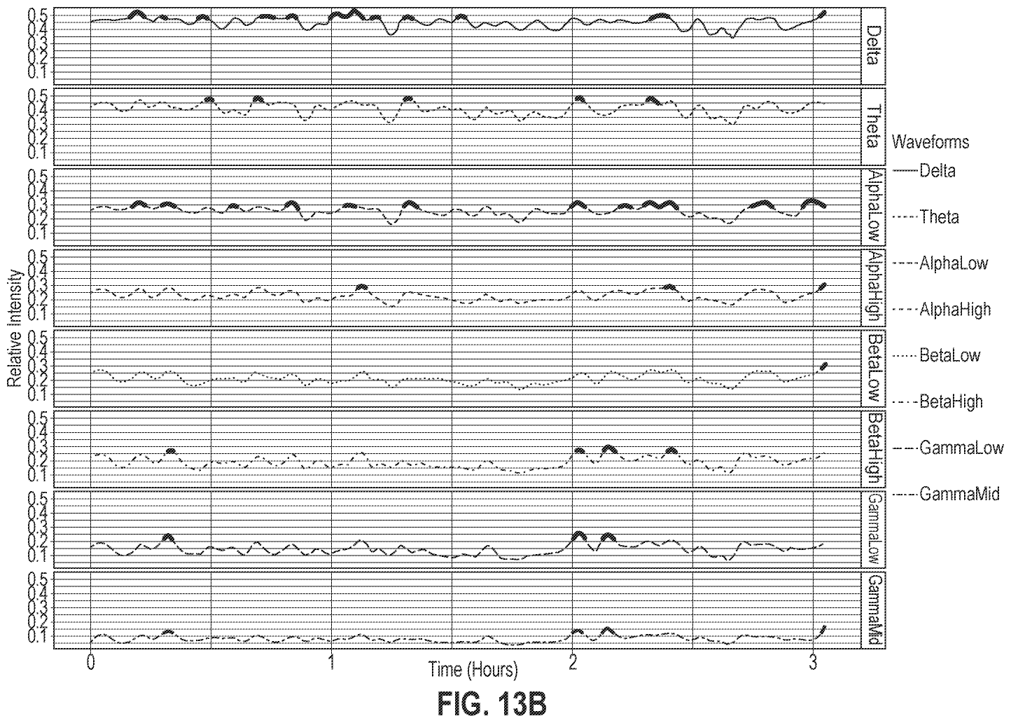

FIGS. 13A and 13B shows neurological test result responses (“Test Results 1”) corresponding respectively to two users (user “A” is FIGS. 13A, user “B” is FIG. 13B) each using a BNMS according to the disclosure. Here, Time is indicated in Hours on the X-axis and Relative Intensity is shown on the Y-axis, and non-emphasized (non-bold) regions indicate normal neurological responses while regions emphasized in bold indicate an increased frequency response in the waveform and may be considered a stressful event. More particularly, the resulting neurological responses for stressful and non-stressful events were collected over a three-hour period for the two individuals A and B with each row representing separate EEG waveforms, Delta, Theta, Alpha Low, Alpha High, Beta Low, Beta High, Gamma Low, and Gamma Mid, respectively.

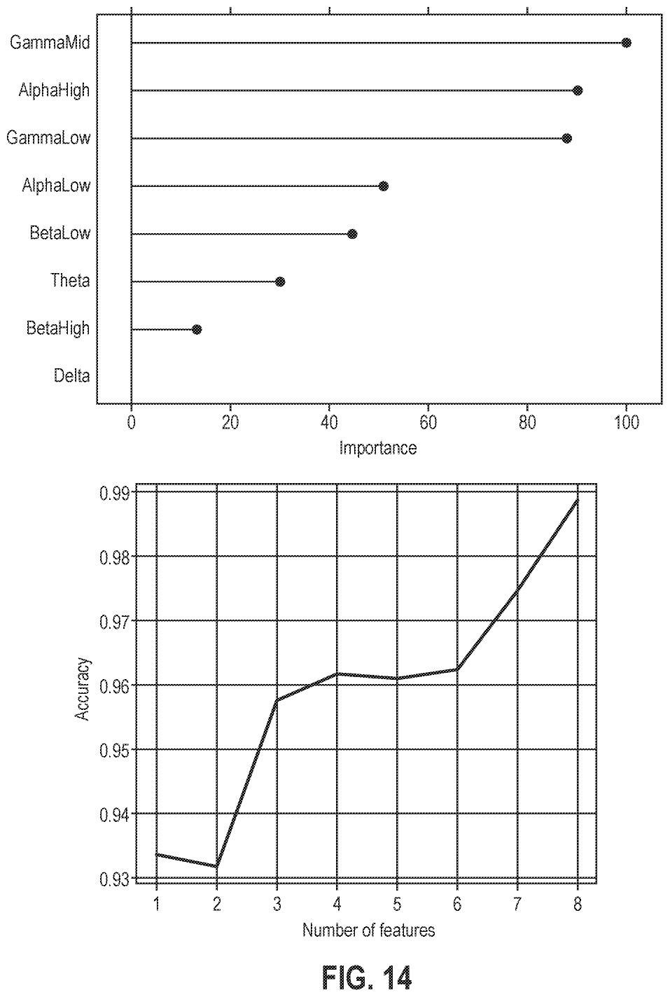

FIG. 14 are “Test Results 2,” which presents methods for determining which biopotential waveforms are most significant for predicting stressful events against normal neurological responses. The uppermost graph shows the “Importance” (X-axis) of each biopotential waveform (Y-axis) with respect to the decision logic of creating machine learning models. Principle Component analysis was used to determine the number of biopotential waveforms necessary to make accurate decisions in the uppermost graph, while the bottom chart shows that with at least four waveforms, models achieved 96% or greater accuracy. The results of dimension reduction methods further revealed that very accurate models can be achieved while simultaneously reducing collected data.

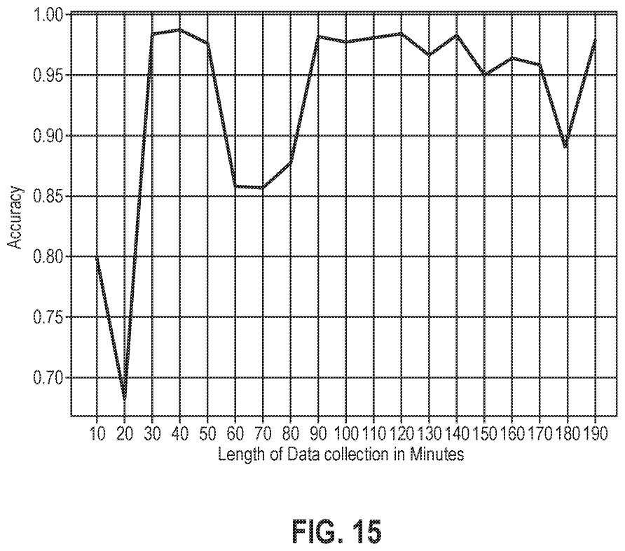

FIG. 15 shows “Test Results 3,” a summary of Artificial Neural Network (ANN) model accuracy over a 3.5 hour duration of data collection (Length of Data Collection (minutes) is shown on the X-axis, and Accuracy (percentage) is on the Y-axis). ANN models were created in ten-minute intervals and prediction accuracy was recorded. These results indicated that optimal accuracy can be achieved after ninety-minutes of collecting individual neurological responses, thereby reducing by more than 50% the amount of data needed for memory storage.

Methods for predicting stressful events based on a user's neurological responses collected from EEG biopotential data are shown in the following table labeled “Test Results 4.”

### Test Results 4

| | Training | Prediction | | | | | Prediction |
|---|---|---|---|---|---|---|---|
| Model | Accuracy | Accuracy | 95% CI | | Sensitivity | Specificity | Kappa |
| Artificial | 98.2% | 98.2% | 97.7% | 98.6% | 98.8% | 97.1% | .961 |
| Neural Network | | | | | | | |
| Support Vector | 95.4% | 95.3% | 94.6% | 96.0% | 96.3% | 93.6% | .898 |
| Machine | | | | | | | |
| Naïve Bayes | 92.9% | 93.3% | 92.4% | 94.1% | 92.0% | 95.7% | .856 |
| Random Forest | 99.7% | 99.8% | 99.7% | 99.9% | 99.9% | 99.7% | .997 |
| Logistic | 99.8% | 99.8% | 99.6% | 99.9% | 99.9% | 99.5% | .996 |
| Regression | | | | | | | |

The Test Results 4 data resulted from training different machine learning models using EEG data collected from individual neurological responses. Here, eight biopotential waveforms were used to create the models. An industry standard 70/30 split was used for training and testing respectively with cross validation to prevent bias. Training accuracies were reported using 70% of the data with Logistic Regression of 99.8% accuracy. Prediction Accuracy, the 95% confidence interval, sensitivity, specificity, and Kappa score are all reported using the 30% remaining held-out data. The Random Forest and Logistic Regression models both reported similar results with 99.8% accuracy. All models demonstrated an impressive ability-greater than 93% accuracy-to use neurological responses to predict stressful events.

Having described various embodiments of the disclosure, examples may include but are not limited to:

EMBODIMENT 1: A method of monitoring biomarkers in an individual, comprising monitoring a monitored individual with at least one sensing device disposed proximate the monitored individual, wherein the sensing device is operable to sense electroencephalographic signals of the monitored individual; sending the electroencephalographic signals to a monitoring system; comparing the electroencephalographic signals against a normalized profile for the monitored individual to assess in real-time whether the monitored individual is in psychological distress, sending an alert from the monitoring system regarding the monitored individual.

EMBODIMENT 2: The method of Embodiment 1, further comprising providing glasses in which the sensing device is disposed.

EMBODIMENT 3: The method of Embodiments 1 or 2, further comprising providing a helmet in which the sensing device is disposed.

EMBODIMENT 4: The method of Embodiments 1, 2, or 3, further comprising providing headgear in which the sensing device is disposed.

EMBODIMENT 5: The method of any of the foregoing embodiments, further comprising providing a vest having a control unit in electrical communication with the sensing device.

EMBODIMENT 6: The method of any of the foregoing embodiments, further comprising providing a vest equipped with a heart rate monitor in electrical communication with the monitoring system.

EMBODIMENT 7: The method of any of the foregoing embodiments, further comprising providing a vest equipped with a respiration monitor in electrical communication with the monitoring system.

EMBODIMENT 8: The method of any of the foregoing embodiments, further comprising providing a module operable to sense a heart rate or respiration.

EMBODIMENT 9: The method of any of the foregoing embodiments, further comprising displaying the electroencephalographic signals in a graphic display.

EMBODIMENT 10: A system for monitoring biomarkers of an individual, comprising a sensing device operable to sense an electroencephalographic signal of a monitored individual; a receiver configured to receive the electroencephalographic signal of the monitored individual; a database for interpreting the electroencephalographic signal received from the receiver; at least one computing device to interpolate the electroencephalographic signal against a normalized profile to determine whether the monitored individual is in biometric danger; and a transmitter configured for sending an alert the monitored individual if the individual is in biometric danger.

EMBODIMENT 11: A method for post-processing biomarker signals collected from a biomonitoring device, the method comprising attaching a biomonitoring device to a subject, the biomonitoring device in communication with biomarkers produced by the subject; obtaining biomarker data from the subject; formatting the biomarker data into storable files; classifying the biomarker data into biopotential waveforms; normalizing the biopotential waveforms; and determining trends from the biopotential waveforms as predictors of neurological stress.

EMBODIMENT 12: The method of Embodiment 11, wherein the biomonitoring device is wearable by the subject.

EMBODIMENT 13: The method of Embodiments 11 or 12, wherein the biomarker data includes EEG waveforms.

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

1. A method of monitoring biomarkers in an individual, comprising:
monitoring a monitored individual with at least one sensing device disposed proximate the monitored individual, wherein the sensing device is operable to sense electroencephalographic signals of the monitored individual;
sending the electroencephalographic signals to a monitoring system;
comparing the electroencephalographic signals against a normalized profile for the monitored individual to assess in real-time whether the monitored individual is in psychological distress; and
if the monitored individual is in psychologic distress, sending an alert from the monitoring system regarding the monitored individual.

2. The method of claim 1, further comprising providing glasses in which the sensing device is disposed.

3. The method of claim 1, further comprising providing a helmet in which the sensing device is disposed.

4. The method of claim 1, further comprising providing headgear in which the sensing device is disposed.

5. The method of claim 1, further comprising providing a vest having a control unit in electrical communication with the sensing device.

6. The method of claim 5, wherein the vest is equipped with a heart rate monitor in electrical communication with the monitoring system.

7. The method of claim 5, wherein the vest is equipped with a respiration monitor in electrical communication with the monitoring system.

8. The method of claim 1, further comprising a module operable to sense a heart rate.

9. The method of claim 1, further comprising a module operable to sense respiration.

10. The method of claim 1, further comprising displaying the electroencephalographic signals in a graphic display.

11. A system for monitoring biomarkers of an individual, comprising:
a sensing device operable to sense an electroencephalographic signal of a monitored individual;
a receiver configured to receive the electroencephalographic signal of the monitored individual;
a database for interpreting the electroencephalographic signal received from the receiver;
at least one computing device to interpolate the electroencephalographic signal against a normalized profile to determine whether the monitored individual is in neuropsychological distress biometric danger; and
a transmitter configured for sending an alert to the monitored individual if the individual is in neuropsychological distress.

12. A method for post-processing biomarker signals collected from a biomonitoring device, the method comprising:
attaching a biomonitoring device to a subject, the biomonitoring device in communication with biomarkers produced by the subject;
obtaining biomarker data from the subject;
formatting the biomarker data into storable files;
classifying the biomarker data into biopotential waveforms;
normalizing the biopotential waveforms; and
determining trends from the biopotential waveforms as predictors of stress.

13. The method as in claim 12, wherein the biomonitoring device is wearable by the subject.

14. The method as in claim 12, wherein the biomarker data includes EEG waveforms.
